(19) United States
(12) Patent Application Publication
(10) Pub. No.: US 2007/0100288 A1
(43) Pub. Date: May 3, 2007

(54) Auto-injection devices and methods for intramuscular administration of medications

(75) Inventors: William Bozeman, Winston-Salem, NC (US); Robert C. Luten, Jacksonville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: UNIVERSITY OF FLORIDA RESEARCH FOUNDATION, INC., Gainesville, FL (US)

(21) Appl. No.: 10/559,422

(22) PCT Filed: Jun. 7, 2004
(86) PCT No.: PCT/US04/17959

§ 371 Date: Sep. 14, 2006

Related U.S. Application Data
(60) Provisional application No. 60/476,365, filed on Jun. 5, 2003.

(51) Int. Cl. A61M 3/00; A61M 5/00; A61M 31/00

(52) U.S. Cl. 604/181; 604/189; 604/506

(57) ABSTRACT

The present invention provides auto-injection devices for the administration of appropriate medication dosage and needle-depth penetration based on a patient's relevant parameters. In one embodiment, the auto-injection device of the present invention comprises non-volumetric indicia based upon a patient parameter for use in selecting the appropriate medicament dosage for administration to the patient. In another embodiment, the non-volumetric indicia not only provides appropriate medicament dosage but also appropriate needle depth penetration.

### CROSS-REFERENCE TO A RELATED APPLICATION

This application claims the benefit of U.S. provisional patent application Ser. No. 60/476,365, filed Jun. 5, 2003.

### BACKGROUND OF INVENTION

There are a variety of injection devices available for delivering medication to a patient, the most typical device being the syringe. With traditional syringes, a user must calculate the correct amount of medication needed for the patient based on a number of parameters (i.e., age, height, and Weight) as well as the correct needle size and depth necessary for appropriate injection of medication into the patient. Once the appropriate dosage and needle requirements have been calculated, the user attaches the appropriate needle to the syringe and inserts the needle into a separate medication vial to withdraw an appropriate medication dose. Once the medication is withdrawn from the vial, the user removes any air bubbles and extra medication, and then injects the medication into the patient at an appropriate depth.

Unfortunately, medication error and/or adverse drug events may result due to errors in professional practice/judgment, health care products, procedures, and systems including, for example, errors in prescribing, order communication, product labeling, packaging, and nomenclature. Medication errors or adverse drug events can include noxious and undesired effect of a drug due to inappropriate medication dosage.

Several studies have demonstrated the high incidence of medication errors and the sometimes fatal results. For example, a death certificate study found a greater than two-fold increase in deaths caused by medication errors between 1993, during which 7,391 people died, and 1983, when 2,876 patients died from medication errors, see, Phillips, D. P., et al., “Increase in U.S. Medication-Error Deaths Between 1983 and 1993,” Lancet, (1998), 351(9103). Another report estimates 6.5 adverse drug events per 100 admissions, as well as an additional cost of $2,000 per adverse drug event, for a hospitalized patient, excluding malpractice costs or the cost of injury to the patient. Furthermore, while most adverse drug events result from errors at the ordering stage, many occurred at the administering stage. See Bates, D. W., et al., “Incidence of Adverse Drug Events and Potential Adverse Drug Events,” JAMA, (1995), 274(1):29-30.

With regard to pediatric treatments, a four-year study that investigated patterns of medication errors in neonatal and pediatric intensive care-units found an error rate of 14.7% with one medication error occurring for every 6.8 admissions. The study found that while the percentage breakdown varied, all health care providers: physicians, nurses, and pharmacists, were responsible, see Raju T N K, et al., “Medication Errors in neonatal and pediatric intensive care units,” Lancet, (1989), 374-376.

Thus, typical syringes, which require a user to calculate the correct amount of medication needed for a patient based on a number of parameters (i.e., age, height, and weight), do not aid in reducing the incidence of medication errors or adverse drug events. Further, these syringes suffer from many drawbacks. For instance, since they are not typically preloaded with medication, the user must carry a separate medication vial. Moreover, people with dexterity disorders often have difficulty lining up the needle portion of the syringe with the rubber septum on the medication vial. This can lead to unintentional needle pricks or excessive time being required to complete an injection, both of which tend to inhibit compliance with a medical regimen. Also, it is often difficult for children or people with failing eyesight to line up the medication with the proper dosage line on the outer casing of the syringe.

As an alternative, automatic injection apparatuses have been developed. An automatic injection enables an individual to self-administer a dosage of liquid medication into his or her flesh. The advantage of automatic injectors is that they contain a measured dosage of a liquid medication in a sealed sterile cartridge, which can be utilized for delivering the medication into the flesh during emergency situations (i.e., such injectors can carry antidotes for nerve gas, insulin for diabetes, or epinephrine for allergic reactions). Another advantage of automatic injectors is that the self-administration of the medication is accomplished without the user initially seeing the hypodermic needle through which the medication is delivered and without having the user manually force the needle into his or her own flesh. Examples of such known injectors are disclosed in U.S. Pat. Nos. 5,085,641; 5,540,664; 5,569,192; and 5,092,843.

A typical drawback of automatic injectors is that they administer a single, one-time dose of medication and are not re-usable. After this single use, the entire apparatus is discarded. This results in high cost and waste of medical equipment.

Another drawback is the relatively short storage life of some medications. The storage life of a medication is generally less than the useful life of the automatic injection apparatus. Automatic injectors can be stored for long periods of time, often 5 years or more. Unfortunately, many medications do not have a comparable storage life. For example, some medications have storage lives of 1-2 years or less. Thus, the medicine could become ineffective before the injector is used, resulting in the wasteful disposal of unused injection apparatuses. This also contributes to high costs.

Even with the means for automatic injection, many of these injection devices still require the user to measure dosage and needle injection depth, which is time consuming and requires an appropriate knowledge base and proper instruments and accessories. Moreover, most automatic injection devices are designed for single use administration to an adult. In emergent situations, it is not feasible to use injection devices of this type on all patients. For example, where a toxic agent has been released, providing an adult dosage amount of an antidote to toxic agents to a small child could harm or even kill the child. Thus, current auto-injection devices would not be available as treatment for about 20% of the current population (children account for roughly 20% of the population) during emergent situations.

Accordingly, it is highly desirable that an injection device be provided that can automatically, efficiently, and appropriately administer medicaments, and which can easily and rapidly be adjusted to administer the appropriate dosage to any patient. Many past devices have failed to provide convenience, accuracy, and efficiency in delivering medicaments to patients of various sizes and ages. Thus, current injection devices have been less than satisfactory.

### BRIEF SUMMARY

The present invention provides auto-injection devices for administration of medication to a patient or via an intravenous line. The devices of the subject invention have a dosage adjusting means and a needle depth adjusting means. In one embodiment, the auto-injection device also has an adjustment mechanism that simultaneously sets the dosage adjusting means and the needle depth adjusting means based on certain patient parameters. These parameters could include, for example, the age, size, weight, and gender of the patient. A particularly advantageous aspect of the devices and methods of the subject invention is their ease of use with both adult and pediatric patients.

According to the present invention, a variety of medicaments can be administered using the subject devices. For example, liquid medications can be integrally stored within the device interior and subsequently administered. Alternatively, the device of the subject invention can readily deliver medications that are provided in solid formulations that require a solvent.

In one embodiment of the present invention, an auto-injection device has a dosage adjusting means, an/or a needle depth adjusting means, an adjustment mechanism, and a reconstitution mechanism. The auto-injection device also has a means for separating different substances from contact until such time as needed for administration. At such time, the reconstitution mechanism is activated to mix the different substances together and allow for proper medication dosage and injection. The auto-injection device has two chambers created and separated by a dividing piston that includes upper and lower plungers. Distally, the lower plunger seals the upper or liquid chamber. The lower or dry drug chamber can be separated from the diluent liquid by an internal hydrophobic membrane that allows air but not water to pass through it.

In use, after removal of the auto-injection device from its packaging, the operator, which may be a patient, can manipulate the adjustment mechanism to select the appropriate indicia of dosage and needle depth. The readily recognizable indicia are based on, and correspond to, an individual's relevant parameters (i.e., gender, age, height, or weight). The indicia are not volumetric. In other words, unlike a standard syringe, which would typically show milliliters, the devices of the subject invention give an indication of dosage and/or needle depth. Thus, the non-volumetric indicia listed on the auto-injection device correspond to appropriate dosages and/or needle injection depth based on the patient's characteristics. The indicia may be based on colors, numbers, or some other easily-identifiable system.

The adjustment mechanism can utilize a variety of known adjustors including, but not limited to, a dial or slide. Concurrent with the movement of the adjustment mechanism, an indicator provides notice to the user of the non-volumetric indicia chosen for the appropriate dosage and/or needle injection depth to be administered.

In a specific embodiment, the indicator is located within a slot in the side of the auto-injection device. Disposed along the sides of the slot are indicia indicating dosage and needle-depth selection based on specific patient parameters. Contemplated indicia include, but are not limited to, a color-coded measurement system. An example of a color-coded measurement system includes a Broselow-Luten tape.

One embodiment of the subject invention provides a method for using an auto-injection device having an adjustment mechanism that adjusts the amount of medicament to be delivered based on specific patient parameters (i.e., patient size). The device is set for an individual's parameter and a protective cover, if used, is removed. The distal end of the device is applied to an appropriate body area for intramuscular injection of the medicament. In a preferred embodiment, the subject device is used to administer a medicament to the thigh or gluteal muscle of a patient. Firm pressure is applied to the auto-injection device so that a trigger, located in the distal end of the device, is actuated. Actuation of the distal end of the device in turn actuates a spring-loaded injector mechanism to advance a sterile needle into the patient's body and to inject the chosen volume of liquid medication. In another embodiment, a trigger located at the proximal end of the device can actuate the spring-loaded injector mechanism to advance the needle into the patient's body and inject the chosen volume of medication.

In another embodiment, the injection device of the subject invention can administer proper medication dosage to an intravenous line.

In another embodiment, the adjustment mechanism adjusts the depth of the needle penetration based on specific patient parameters (i.e., patient age). In yet another embodiment, the adjustment mechanism simultaneously adjusts the amount of medicament delivered and the depth of the needle penetration based on specific patient parameters (i.e., patient size and age).

Another embodiment of the subject invention provides an adjustment mechanism that can be dialed up or down without limitation. In another embodiment, the auto-injection device of the subject invention has a means for locking the adjustment mechanism in a selected position either before or when the trigger of the device is actuated and injection of the medicament into the patient occurs.

A further embodiment provides a device for pediatric patients requiring medicaments prior to the establishment of intravenous access.

### BRIEF DESCRIPTION OF DRAWINGS

FIG. 1 is a schematic diagram of an injection device for administering liquid medicaments in accordance with the present invention.

FIG. 2 is a longitudinal section view of the device shown in FIG. 1.

FIG. 3 is a schematic diagram of an auto-injection device for dispensing reconstituted dry medicaments in accordance with the present invention.

FIG. 4 is a longitudinal section view of the device shown in FIG. 3.

### DETAILED DISCLOSURE

The present invention provides auto-injection devices for administration of medication. In a preferred embodiment, the auto-injection device has a dosage adjusting means, a needle depth adjusting means, and an adjustment mechanism. These aspects of the invention provide an auto-injection device that easily and automatically administers the appropriate medication dose and/or needle-depth penetration to all patients, including adults and children, based on specific patient parameters.

In a preferred embodiment, the adjustment mechanism simultaneously establishes the setting for the dosage and the needle depth. A variety of medicaments can be administered using the subject device, including solid formulations/solvent or liquid medications integrally stored within the device interior.

In use, after removal of the auto-injection device from its packaging, the operator, which may be a patient, can actuate the adjustment mechanism to select the appropriate settings (as shown by easily recognized non-volumetric indicia) that corresponds to an individual patient's relevant parameter(s) (i.e., age, height, weight). The adjustment mechanism can utilize a variety of known adjustors including, but not limited to, a dial or slide. In one embodiment, concurrent with the movement of the adjustment mechanism, an indicator moves within a slot in the side of the auto-injection device. Disposed along the sides of the slot are indicia representing the appropriate dosage and needle-depth selection for an individual patient. Contemplated indicia include, but are not limited to, a color-coded measurement system. An example of a color-coded measurement system includes a Broselow-Luten tape.

The terms “non-volumetric indicia” or “parameter indicia” as used herein, refer to displays/markings that represent the amount of medication to be administered and/or the appropriate needle-depth penetration. Non-volumetric indicia (as opposed to known volumetric indicia, i.e., mL) can include numbers, characters, or colored markers. For example, non-volumetric indicia can include a series of different colors (i.e., red, orange, yellow). Each of the colors represents the appropriate needle-depth penetration and/or amount of medication that should be administered to an individual based on the individual's parameters (i.e., age, height, weight, or gender).

The term “individual” or “patient” includes animals of avian, mammalian, or reptilian origin. Mammalian species that can benefit from the methods and devices of the subject invention include, but are not limited to, apes, chimpanzees, orangutans, humans, monkeys; dogs, cats, guinea pigs, and mice.

In one embodiment, an auto-injection device of the present invention includes an adjustment mechanism that adjusts the amount of medicament to be delivered based on an individual patient's parameters (i.e., patient size). In use, the device is set for the individual's parameter and a protective cover, if used, is removed. The distal end of the device is applied to an appropriate body area for intramuscular injection of the medicament. In a preferred embodiment, the subject device is used to administer a medicament to the thigh or gluteal muscle of a patient. Firm pressure is then applied to the auto-injection device so that a trigger, located in the distal end of the device, is actuated. Actuation of the distal end of the device in turn actuates a spring-loaded injector mechanism to advance a sterile needle into the patient's body and to inject the chosen volume of liquid medication.

In another embodiment, the injection device of the subject invention is used to administer the proper dosage of medication to an intravenous line.

In another embodiment, a trigger located at the proximal end of the device can actuate the spring-loaded injector mechanism to advance the needle into the patient's body and inject the chosen volume of medication.

Another embodiment of the subject invention has an adjustment mechanism that adjusts the depth of the needle penetration based on an individual's parameters (i.e., patient age). Again, after the device has been set for the individual's parameter, a protective cover, if used, is removed and the distal end of the device is applied to an appropriate body area for intramuscular injection of the medicament, wherein the needle depth penetration is appropriate based on the patient's parameter.

In yet another embodiment, the adjustment mechanism simultaneously adjusts the amount of medicament delivered and the depth of the needle penetration based on an individual patient's parameters.

A further embodiment provides an auto-injection device having an adjustment mechanism that can be dialed up or down without limitation. In another embodiment, the auto-injection device of the subject invention has a means for locking the adjustment mechanism in a selected position either before or when the trigger in the proximal end of the device is actuated.

A further embodiment provides a device for pediatric patients requiring medicaments prior to the establishment of intravenous access.

The following examples and accompanying figures describe specific embodiments of the device and methods of the present invention, and features thereof. With regard to means for fastening, mounting, attaching, or connecting the components of the present invention to form the device as a whole, unless specifically described otherwise, such means are intended to encompass conventional fasteners such as threaded connectors, snap rings, clamps such as screw clamps and the like, rivets, toggles, pins, and the like. Components may also be connected by adhesives, glues, welding, ultrasonic welding, and friction fitting or deformation, if appropriate. Unless specifically otherwise disclosed or taught, materials for making components of the present invention may be selected from appropriate materials such as metal, metallic alloys, natural and manmade fibers, vinyls, plastics, and the like, and appropriate manufacturing or production methods including casting, extruding, molding, and machining may be used.

References to front and back, right and left, top and bottom, and upper and lower are intended for convenience of description, not to limit the present invention or its components to any one positional or special orientation.

The auto-injection devices, as disclosed in U.S. Pat. Nos. 6,290,679; 6,193,698; 5,569,192; 5,540,664; 5,141,496; and 5,104,380, may be modified consistent with the teachings provided herein for use according to the subject invention. Specifically, the auto-injection devices of the subject invention have one or more of a dosage adjusting means, a needle depth adjusting means, and an adjustment mechanism

### EXAMPLE 1

### Liquid Medicaments

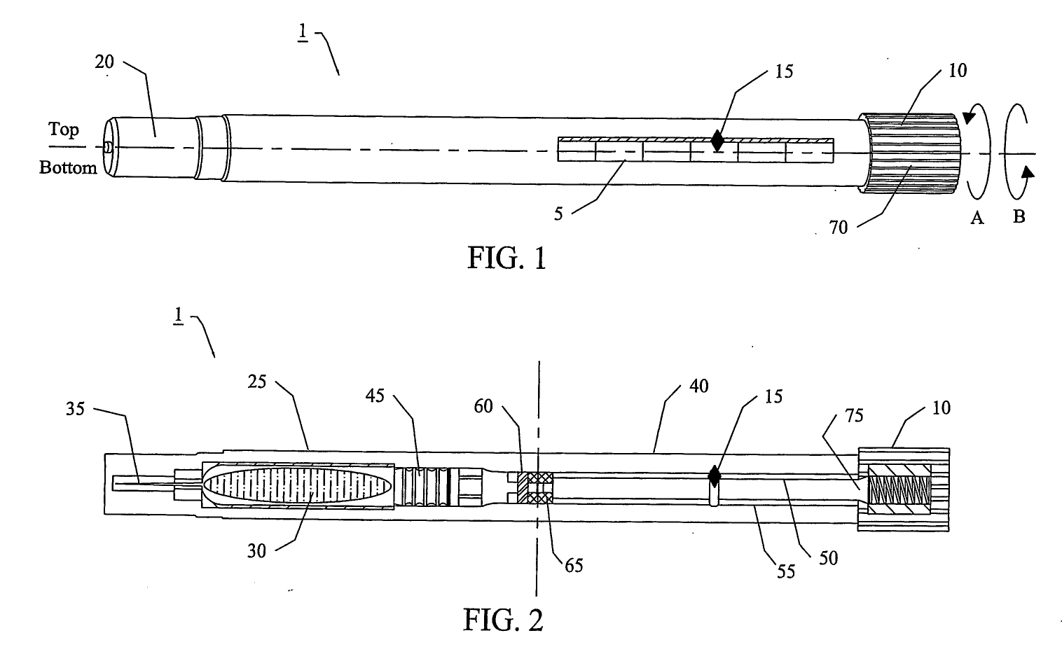

As illustrated in FIG. 1, an auto-injection device 1 in accordance with the present invention is provided. The device 1 has non-volumetric indicia 5 that correspond to medication dosages based on an individual's relevant parameters. The appropriate dosage, which can be adjusted continuously or discretely, are marked on a non-volumetric scale (i.e., Broselow-Luten tape) affixed to the housing of the device 1. By rotating an adjustment knob 10 around its longitudinal axis, a user can select the appropriate dose of medication to be administered and/or needle penetration depth based on the patient's relevant parameters. A movable indicator 15 displays to the user a representation of the relevant parameter and appropriate dosage amount to be administered using the subject device 1. The movable indicator 15 corresponds in movement to that of the adjustment knob 10. A needle protector 20 is provided to protect the user from accidental needle punctures.

In this embodiment, the adjustment knob 10 actuates a dosage adjusting means and/or a needle depth adjusting means. In one embodiment, the adjustment knob 10 also operates as the dispensing button. The dosage to be administered and/or needle penetration depth is represented by the indicator 15. The needle depth adjusting means adjusts the depth to which the needle will penetrate the dermis of the patient during administration of the medication using the subject device 1.

FIG. 2 illustrates a longitudinal cross-section of an automatic injection device 1 of the subject invention. The device 1 comprises an elongated housing, including a distal enclosure 25 for accommodating a container of medication 30 and a needle 35, and a proximal enclosure 40. The proximal enclosure 40 accommodates a piston 45, to which is attached the movable indicator 15. Movement of the piston 45 in the direction of the medication container 30 outlet via the needle 35 displaces the medication.

The movement of the piston 45 (and moveable indicator 15) is caused by contact pressure of a threaded rod 50. The threaded rod 50 forms the driven member of a spindle drive, configured to include the threaded rod 50 and a threaded sleeve 55. The threaded sleeve 55 surrounds the threaded rod 50 as the drive member. To move the piston 45, the threaded sleeve 55 together with the threaded rod 50 is advanced against an elastic return force by actuation of the adjustment knob 10. The adjustment knob 10 in turn actuates the piston 45 to advance in the direction toward the distal end of the proximal enclosure 40. The adjustment knob 10, the drive members 50, 55 and the piston 45 are linearly shifted along a common axis, the shifting axis, as indicated as a dot-dash in the figures. In this arrangement, the distance by which the adjustment knob 10 and the drive members 50, 55 are shifted on actuation is always the same. Thus, the distance covered by the piston 45, as influenced by the drive 50, 55, allows for variable selection of a dose of medication to be administered by injection.

The adjustment knob 10 comprises a sleeve part 70, closed off by an exchange part 75. The sleeve part 70 of the adjustment knob 10 protrudes through a proximal enclosure 40. In the region of the sleeve part 70, the adjustment knob 10 is connected to the threaded sleeve 55 by an anti-rotation lock. Thus, rotating the adjustment knob 10 automatically engages in rotation the threaded sleeve 55 around its longitudinal axis. The threaded rod 50 is linearly guided secured against rotation so that a rotation of the threaded sleeve 55 automatically results in a linear shift of the threaded rod 50. At its proximal end facing the piston 45, the threaded rod 50 comprises a flange or plunger 60, with which it advances the piston 45 in the medication container 30 on actuation of the adjustment knob 10. The shifting path of the threaded rod plunger 60 is the same in length for each injection. Advancement is made against the elastic restoring force of a compression spring 65, disposed between an appendage of the proximal enclosure 40 and a corresponding companion appendage on the threaded sleeve 55. The compression spring 65 attempts to push back to its distal position the “actuating means,” essentially consisting of the threaded sleeve 55, threaded rod 50, and the adjustment knob/dispensing button 10.

The adjustment knob 10 comprises axially extending ridges and furrows 70 arranged uniformly distributed about the circumference of an outer shell surface of the adjustment knob 10 to ensure user grip of the adjustment knob 10.

In one embodiment, the non-volumetric parameter indicia correspond to medication dosages and needle penetration depths based on an individual's height. The non-volumetric parameter indicia comprises a variety of colors that are found on a Broselow-Luten tape. The colors on a Broselow-Luten tape correspond to the appropriate medication dosage and needle penetration depth based on an individual's height. By way of example, where the medication is atropine to be used as an antidote to a nerve agent, non-volumetric parameter indicia of the color pink corresponds to the dosage of 0.32 mL of atropine and needle depth of ½ to ⅞ inch for a patient less than 76 cm in height.

In another embodiment, the non-volumetric parameter indicia correspond to medication dosages based on an individual's weight. The non-volumetric parameter indicia comprise a variety of characters corresponding to a dosage based on an individual's weight. By way of example, where the medication is atropine to be used as an antidote to a nerve agent, non-volumetric parameter indicia of 3 kg corresponds to a dosage of 0.12 mL of atropine (IV/IM 0.1 mg/mL concentration) for an individual weighing 3 kg. Non-volumetric parameter indicia of 4 kg correspond to the dosage of 0.2 mL of atropine (IV/IM 0.1 mg/mL concentration) for a patient that weighs 4 kg.

In another embodiment, the non-volumetric parameter indicia correspond to needle penetration depth based on an individual's height. The non-volumetric parameter indicia comprises a variety of colors that are found on a Broselow-Luten tape. The colors on a Broselow-Luten tape correspond to the appropriate needle depth penetration based on an individual's height. By way of example, non-volumetric parameter indicia of the color pink corresponds to the needle depth of ½ to ⅞ inch for a patient less than 76 cm in height.

### EXAMPLE 2

### Solid/Solvent Medicament Mixtures

Another embodiment of the subject invention provides an auto-injection device for dispensing dry or unstable medications that require reconstitution prior to administration to a patient. The auto-injection device, as described in U.S. Pat. Nos. 5,971,953 5,393,326; 4,983,164; 4,413,991; 4,202,314; and 4,214,584, may be modified consistent with the teachings provided herein for use according to the subject invention.

According to the subject invention, the auto-injection device has a dosage adjusting means, and/or a needle depth adjusting means, an adjustment mechanism, and a reconstitution mechanism. The reconstitution mechanism has two chambers created and separated by a dividing piston that includes upper and lower plungers. Distally, the lower plunger seals the upper or liquid chamber. The lower or dry drug chamber can be separated from the diluent liquid by an internal hydrophobic membrane that allows air but not water to pass through it. The auto-injection device of the subject invention is generally cylindrically shaped, having proximal and distal end portions. A middle section of the auto-injection device is of an enlarged diameter.

A bore extends between the proximal and distal end portions, the bore including upper and lower chamber sections for containing medicine contents to be dispensed including an upper liquid component and a lower dry medicinal component. A dispensing needle at the distal end of the housing can be provided for receiving the medicine contents of the lower chamber after mixing.

A pair of pistons is provided that are separately movable. A lower piston occupies the position in between the ends of the auto-injection device, and in between the upper and lower chambers. The lower piston is movable between upper and lower positions. An upper piston is positioned at the proximal end of the housing and slides within the bore during use.

The enlarged diameter middle portion of the barrel of the auto-injection device carries one or a plurality of longitudinally extending channels. These channels are positioned at the middle of the housing and form a connection between the upper and lower chambers. The floating chamber has a maximum sidewall dimension that is less than the length of the channel or channels. The channels can therefore convey fluid in between the proximal and distal ends of the syringe and in between the upper and lower chambers when the first piston occupies a position adjacent the longitudinal channels and the ends of each channel extend beyond the ends of the lower piston.

The lower piston forms a seal to retain the liquid contents of the upper chamber away from the lower chamber when the first piston is in the upper position. The lower piston forms a seal that seals the combined liquid and dry contents from the channels prior to dispensing and after the liquid and dry medicinal portions have been reconstituted.

In accordance with the present invention, a dual chambered auto-injection device contains longitudinally extending bypass channels. These longitudinal channels are part of an enlarged middle diameter section of the auto-injection device, so designed in depth and width as to facilitate thorough mixing of all pharmaceuticals.

The advantage is obtained by the mixing channels being critically placed to begin and end generally equidistant from each end of the syringe so as to permit the lower chamber to accept and instantly retain a predetermined volume of diluent contained and transferred through the bypass channels from the upper chamber.

The dampening slot slows movement of the lower plunger so as to permit complete mixing of the diluent from the upper chamber with the dry medication in the lower chamber. The floating piston is forced from the dampening slot by the abutment of the upper piston against the lower piston. This occurs when all of the diluent fluid between the upper and lower plunger has passed through the ribbed by-pass portals into the lower chamber.

The length of the bypass portals is of any length greater than the length of the dividing piston, but not of such length as to encroach into the distal chamber of the auto-injection device, or of such length as prevent the putative lower chamber from receiving the required volume for exact reconstitution and tight resealing by the dividing piston.

Operationally, the adjustment mechanism is manipulated to select the appropriate settings (as shown be easily recognized non-volumetric indicia) that corresponds to an individual's relevant parameter(s) (i.e., age, height, weight). The adjustment mechanism can utilize a variety of known adjustors including, but not limited to, a dial. Once the device is set for the individual's parameter(s) (i.e., patient size), the reconstitution mechanism is actuated so that the upper plunger pressurizes liquid in the upper chamber, causing the lower plunger to move downward and enter the by-pass mixing portals. The bypass portals, now opened and confluent to each side of the lower plunger, cause accelerated fluid flow from the upper chamber to mix and reconstitute the dry contents in the lower drug chamber. Alternatively, the reconstitution mechanism is actuated first and then the needle depth and/or dosage (based on a patient parameter) is selected.

Then, the distal end of the device of the subject invention is applied to an appropriate body area for intramuscular injection of the reconstituted medicament. In one embodiment, firm pressure applied to the distal end of the auto-injection device actuates a spring-loaded injector mechanism to advance a sterile needle into the patient's body to inject the chosen volume of liquid medicament. Alternatively, a trigger, if provided, located at the proximal end of the device can actuate the spring-loaded injector mechanism to advance a sterile needle into the patient's body to administer the appropriate dosage of medication to the patient.

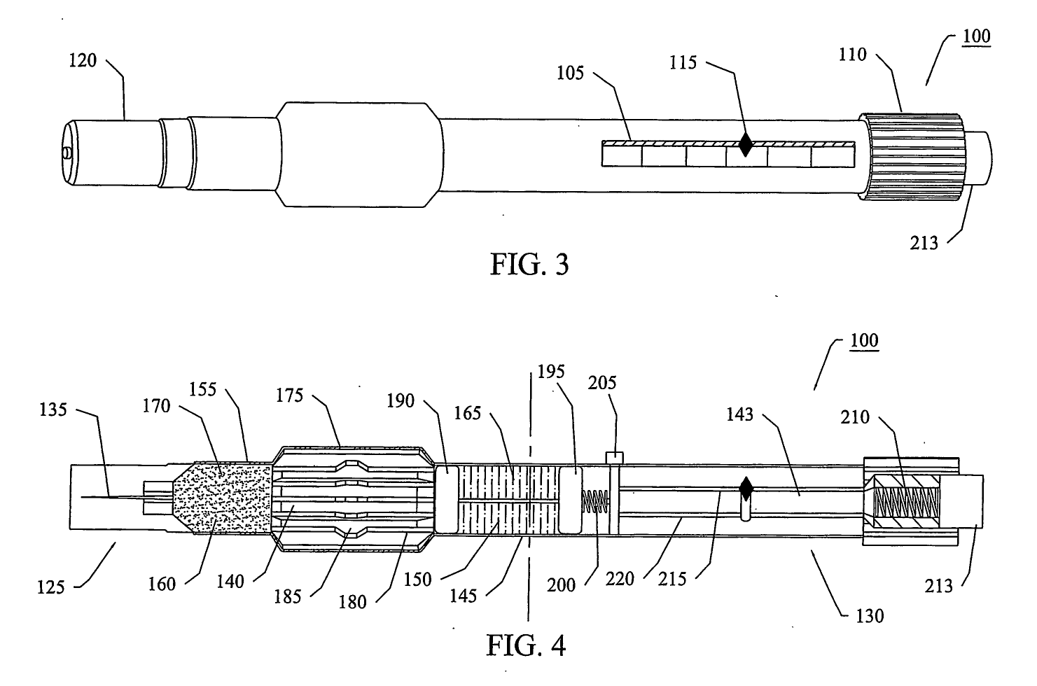

One embodiment of the present invention provides a dual chamber auto-injection device 100, as shown generally in FIGS. 3 and 4. The auto-injection device 100 has non-volumetric indicia 105 that correspond to medication dosages based on an individual's relevant parameters. The appropriate dosage, which can be adjusted continuously or discretely, are marked on a non-volumetric scale (i.e., Broselow-Luten tape) affixed to the housing of the device 100.

By rotating an adjustment knob 110 around its longitudinal axis, a user can select the appropriate dose of medication to be administered and/or needle penetration depth based on the patient's relevant parameters. A movable indicator 115 displays to the user a representation of the relevant parameter and appropriate dosage amount to be administered using the subject device 100. The movable indicator 115 corresponds in movement to that of the adjustment knob 110. A needle protector 120 is provided to protect the user from accidental needle punctures.

The auto-injection device 100 has a central longitudinal bore 140. The bore 140 accommodates a tubular member 143, to which is attached the movable indicator 115. Movement of the tubular member 143 displaces the amount of medication to be reconstituted.

The auto-injection device 100 has a distal end 125 and a proximal end 130. Distal end 125 can be provided with a needle 135 so that liquid contained within the syringe bore 140 can be discharged via the needle 135.

The auto-injection device 100 also has an upper cylindrical section 145 having an upper chamber 150 for containing fluid and a lower cylindrical section 155 with a lower chamber 160. The upper chamber 150 contains a liquid diluent 165. Lower chamber 160 contains a dry medicine or drug 170.

An enlarged diameter section 175 is provided in between the distal 125 and proximal 130 ends. The enlarged diameter section 175 has a cylindrical wall, a frustoconical wall, a second frustoconical wall, and a plurality of radially and longitudinally extending ribs 180.

The ribs 180 have cutouts or recesses 185 that define in combination a dampening slot for receiving the periphery of a lower piston 190. Each pair of ribs 180 defines a bypass flow channel. During use, the lower piston 190 registers in the dampening slot defined by recesses 185 so that the lower piston 190 is held by the recesses 185 until the liquid diluent 165 can flow via the bypass flow channels from upper chamber 150 into lower chamber 160. There, it mixes with the dry drug 170.

An upper piston 195 is positioned within the bore 140 next to the proximal end 125 of the device 100. The lower piston 190 is positioned in between enlarged diameter section 175 and distal end 130. In this fashion, the upper chamber 150 is formed in between lower piston 190 and upper piston 195. The lower chamber 160 is that portion of syringe bore 110 below lower piston 190 or in between lower piston 190 and distal end 125 of the device 100. The liquid diluent 165 contained in upper chamber 150 is separated from and sealed from the dry drug 170 in lower chamber 160.

A first spring-loaded mechanism 200 is used to force the upper piston 195 and lower piston 190 from the proximal end 130 toward the distal end 125 of the device 100 to reconstitute the medicament to be delivered to the patient. The spring-loaded mechanism 200 can be actuated using a variety of known methods including, but not limited to, a release button 205.

Once the spring-loaded mechanism 200 is actuated, the lower piston 190 moves downwardly until the periphery of the lower piston 190 engages the correspondingly shaped recesses 185 of ribs 180, which form a dampening slot to prevent further downward movement of the lower piston 190. Simultaneously, the upper piston 195 also moves downwardly until the upper piston 195 engages the lower piston 190. The upper and lower piston remain engaged until such time as the medicament is to be administered.

When the reconstituted medicament is to be administered, the distal end 125 of the device 100 is applied to the appropriate body area for injection. A trigger 213 located in the proximal end 130 of the device 100, is then depressed to actuate a second spring-loaded mechanism 210 that forces both pistons 190, 195 to move downwardly so that the reconstituted drug product can be dispensed via needle 135 into the patient.

The auto-injection device of the present invention has an adjustment mechanism to adjust the dosage of the reconstituted drug product to be administered to a patient. Contact pressure of a threaded rod 215 causes the proximal end of the tubular member 143 (on which a moveable indicator 115 is located) to move toward the distal end 125 of the device. The threaded rod 215 forms the driven member of a spindle drive, configured to include the threaded rod 215 and a threaded sleeve 220. The threaded sleeve 220 surrounds the threaded rod 215 as the drive member. To move the piston tubular member 143, the threaded sleeve 220 together with the threaded rod 215 is advanced against an elastic return force by actuation of the adjustment knob 110. The adjustment knob 110 in turn actuates the tubular member 143 to advance in the direction toward the distal end of the proximal enclosure 130. The adjustment knob 110, the drive members 215, 220, and the tubular member 143 are linearly shifted along a common axis, the shifting axis, as indicated as a dot-dash in the figures. In this arrangement, the distance by which the adjustment knob 110 and the drive members 215, 220 are shifted on actuation is always the same. Thus, the distance covered by the tubular member 143, as influenced by the drive 215, 220, allows for variable selection of a dose of medication to be administered by injection.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. An auto-injection device comprising non-volumetric indicia for at least one patient parameter, an indicator, a dosage adjusting means, and an adjustment mechanism.

2. The auto-injection device of claim 1, wherein the adjustment mechanism sets the dosage adjusting means based on the patient parameter.

3. The auto-injection device of claim 1, further comprising a needle depth adjusting means.

4. The auto-injection device of claim 3, wherein the adjustment mechanism simultaneously sets the dosage adjusting means and the needle depth adjusting means based on a patient parameter.

5. The auto-injection device of claim 2, wherein the patient parameter is selected from the group consisting of age, size, height, weight, and gender of the patient.

6. The auto-injection device of claim 5, wherein the patient parameter is age and the patient is a pediatric patient.

7. The auto-injection device of claim 1, wherein the non-volumetric indicia is selected from the group consisting of numbers, characters, and colors.

8. The auto-injection device of claim 1, wherein the non-volumetric indicia is a Broselow-Luten tape.

9. The auto-injection device of claim 1, further comprising a means for separation, and a reconstitution mechanism, wherein the means for separation separates different substances from contact with each other until such time as needed for administration.

10. The auto-injection device of claim 9, wherein the means for separation is two chambers created and separated by a dividing piston, wherein the dividing piton includes upper and lower plungers.

11. A method for injecting medication to a patient comprising:
a) assessing a patient parameter;
b) manipulating an adjustment mechanism on an auto-injection device comprising medicament, a distal end, a proximal end, a dosage adjusting means, non-volumetric indicia, an indicator, and the adjustment mechanism, wherein said non-volumetric indicia is based upon the patient parameter;
c) assessing indicator position against the non-volumetric indicia as the adjustment mechanism is manipulated;
d) once the indicator is positioned on the appropriate non-volumetric indicia based on the patient parameter, administering the medicament to the patient by actuating the auto-injection device to release the medicament out of the distal end of the device.

12. The method of claim 11, wherein the step of administering medicament to the patient comprises actuating the auto-injection device to release the medicament out of the distal end of the device to an intravenous line.

13. The method of claim 11, wherein the step of administering medicament to the patient comprises actuating the auto-injection device to release the medicament out of the distal end of the device into a body of the patient.

14. The method of claim 11, wherein the patient parameter is selected from the group consisting of age, size, height, weight, and gender of the patient.

15. The method of claim 11, wherein the non-volumetric indicia is selected from the group consisting of numbers, characters, and colors.

16. The method of claim 15, wherein the non-volumetric indicia is a Broselow-Luten tape.

17. The method of claim 11, wherein the auto-injection device further comprises a needle depth adjusting means, wherein manipulating the adjustment mechanism simultaneously sets the dosage adjusting means and the needle depth adjusting means based on the patient parameters.

18. The method of claim 11, wherein the auto-injection device further comprises a means for separation, a reconstitution mechanism, and at least two different substances that form the medicament, wherein the means for separation prevents the substances from contact with each other until such time as needed for administration.
